(19) United States
(12) Patent Application Publication
(10) Pub. No.: US 2026/0053667 A1
(43) Pub. Date: Feb. 26, 2026

(54) INTRAOCULAR PRESSURE ADJUSTMENT BASED ON ESTIMATED PATIENT EYE LEVEL DISTANCE

(71) Applicant: Alcon Inc., Fribourg (CH)

(72) Inventors: Michael Sam Cardamone, Keller, TX (US); Paul R. Hallen, Colleyville, TX (US)

(21) Appl. No.: 19/283,419

(22) Filed: Jul. 29, 2025

Related U.S. Application Data
(60) Provisional application No. 63/685,334, filed on Aug. 21, 2024.

(51) Int. Cl. A61F 9/007; G01S 7/481; G01S 7/51; G01S 17/89

(52) U.S. Cl. CPC A61F 9/007; G01S 7/4813; G01S 7/51; G01S 17/89

(57) ABSTRACT

A surgical system includes at least one of an infusion subsystem or an aspiration subsystem, a distance estimation device, and one or more processors. The distance estimation device is configured to estimate a vertical distance between a point of reference associated with the surgical system and a patient eye level (PEL) using light. The point of reference corresponds to a pressure level setting for infusion or aspiration. The one or more processors are configured to receive the vertical distance and cause the at least one of the infusion subsystem or the aspiration subsystem to control an infusion pressure or an aspiration pressure based on the vertical distance to match the pressure level setting at the PEL.

### INTRODUCTION

Maintaining intraocular pressure (IOP) within a desired range is an important factor during ophthalmic surgical interventions including, for example, refractive surgery, lens-replacement surgery, and retinal surgery. During surgical interventions, an ophthalmic surgical system uses infusion pressure to deliver an irrigation fluid into the interior of the eye, and the ophthalmic surgical system uses aspiration (vacuum) pressure to remove vitreous and lens fragments from the interior of the eye. The infusion pressure and the aspiration pressure dictate the resultant IOP.

### SUMMARY

Aspects of the present disclosure relate to surgical systems for ophthalmic (eye) procedures, and more specifically, to infusion and aspiration systems for performing ophthalmic procedures.

In certain embodiments, a surgical system includes at least one of an infusion subsystem or an aspiration subsystem, a distance estimation device, and one or more processors. The distance estimation device is configured to estimate a vertical distance between a point of reference associated with the surgical system and a patient eye level (PEL) using light. The point of reference corresponds to a pressure level setting for infusion or aspiration. The one or more processors are configured to execute instructions that cause the one or more processors to receive the vertical distance and cause the at least one of the infusion subsystem or the aspiration subsystem to control an infusion pressure or an aspiration pressure based on the vertical distance to match the pressure level setting at the PEL.

In certain embodiments, a method includes estimating, by a distance estimation device, a vertical distance between a patient eye level (PEL) and a point of reference associated with a surgical system. The point of reference is disposed above or below the PEL and the vertical distance is estimated using light. An amount to increase or an amount to decrease an infusion pressure or an aspiration pressure to match a pressure level setting is computed by one or more processors based on the vertical distance. The infusion pressure or the aspiration pressure is modified by at least one of an infusion subsystem or aspiration subsystem based on the amount to increase or the amount to decrease.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates an example surgical environment, according to embodiments described herein.

FIG. 2A illustrates a first example system for estimating a distance, according to embodiments described herein.

FIG. 2B illustrates a first example user interface, according to embodiments described herein.

FIG. 3A illustrates a second example system for estimating a distance, according to embodiments described herein.

FIG. 3B illustrates a second example user interface, according to embodiments described herein.

FIG. 4 illustrates a third example user interface, according to embodiments described herein.

FIG. 5A illustrates an example of a surgical console, according to embodiments described herein.

FIG. 5B illustrates a first example of a distance estimation system, according to embodiments described herein.

FIG. 5C illustrates a second example of a distance estimation system, according to embodiments described herein.

FIG. 6 illustrates an example method for adjusting infusion pressure or aspiration pressure, according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to surgical systems for ophthalmic (eye) procedures, and more specifically, to infusion and aspiration systems for performing ophthalmic procedures.

The designations “first” and “second” as used herein are not meant to indicate or imply any particular positioning or other characteristic. Rather, when the designations “first” and “second” are used herein, they are used only to distinguish one component from another. The terms “attached,” “connected,” “coupled,” and the like mean attachment, connection, coupling, etc., of one part to another either directly or indirectly through one or more other parts, unless direct or indirect attachment, connection, coupling, etc., is specified.

In order to control the IOP, an infusion pressure level and an aspiration pressure level are provided to the ophthalmic surgical system by a user. The surgical system includes a point of reference which indicates a calibrated elevation at which delivered infusion and aspiration pressure match the user specified infusion and aspiration pressure. Because of gravity, if a patient eye level (PEL) is above the point of reference, then the infusion pressure delivered at the PEL will be less than the user specified infusion pressure and the aspiration pressure delivered at the PEL will be greater than the user specified aspiration pressure. Conversely, if the PEL is below the point of reference, then the infusion pressure delivered at the PEL will be greater than the user specified infusion pressure and the aspiration pressure delivered at the PEL will be less than the user specified aspiration pressure. As used herein, the terms “infusion” and “irrigation” are used interchangeably to denote positive pressure as opposed to aspiration or vacuum pressure. Although the term “infusion” is more commonly used in the context of posterior segment procedures and the term “irrigation” is more commonly used in the context of anterior segment procedures, it is to be appreciated that, as used herein, the terms “infusion” and “irrigation” are interchangeable and the terms are not intended to be limited to a particular type of ophthalmic procedure.

The ophthalmic surgical system compensates for the differences between the specified and delivered pressures by receiving a vertical distance between the PEL and the point of reference as an input from the user. However, the vertical distance is difficult to accurately measure because the PEL and the point of reference are not included in the same vertical plane. If an inaccurate vertical distance is provided to the ophthalmic surgical system, then the specified and delivered pressures will not be the same, which is undesirable.

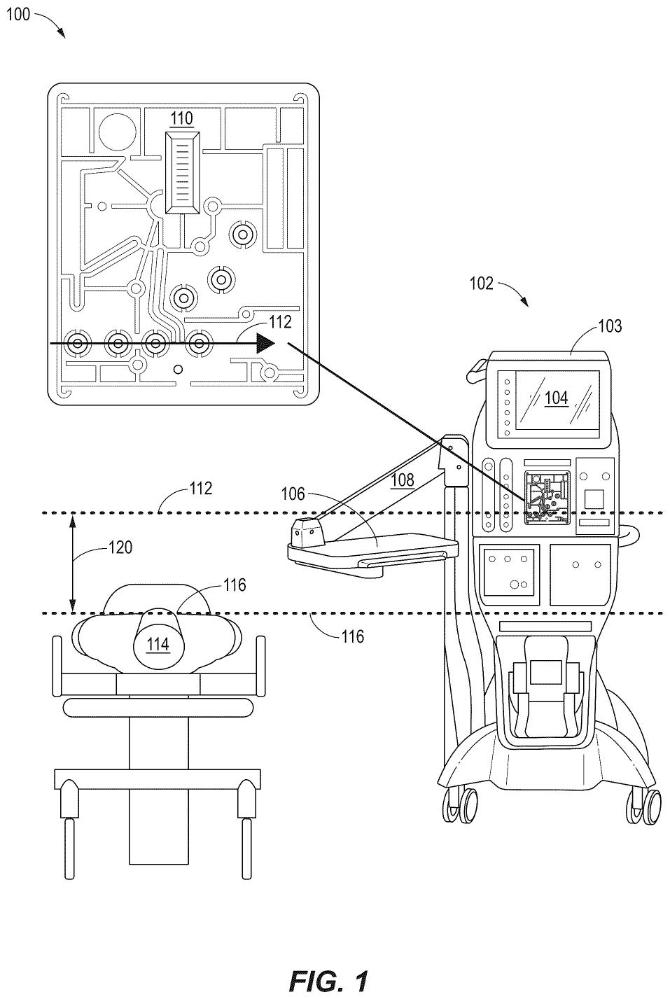

FIG. 1 illustrates an example surgical environment 100, according to embodiments described herein. As shown, the surgical environment 100 includes an ophthalmic surgical system 102. The ophthalmic surgical system 102 is illustrated to include a console 103, a display device 104, and a tray 106 coupled to the console 103 by a mechanical arm 108. In some embodiments, the ophthalmic surgical system 102 includes a cassette 110 which indicates a point of reference 112 for infusion and aspiration pressure supplied by the ophthalmic surgical system 102. The point of reference 112 corresponds to an elevation from the ground at which the infusion and aspiration pressure supplied matches infusion and aspiration pressure levels specified by a user of the ophthalmic surgical system 102.

Consider an example in which the user interacts with a user interface displayed by the display device 104 to specify an infusion (irrigation) pressure of 25 mmHg (millimeters of mercury) and an aspiration (vacuum) pressure of 150 mmHg. In this example, if the infusion and aspiration are delivered at an elevation that is above the point of reference 112, then the delivered infusion (irrigation) pressure will be less than 25 mmHg and the delivered aspiration (vacuum) pressure will be greater than 150 mmHg due to the force of gravity (e.g., the aspiration zero set point will be offset by the equivalent gravity amount). Accordingly, the force of gravity is subtractive to the infusion pressure and additive to the aspiration pressure for the delivery elevation that is above the point of reference 112. Conversely, if the infusion and aspiration are delivered at an elevation that is below the point of reference 112, then the delivered infusion (irrigation) pressure will be greater than 25 mmHg and the delivered aspiration (vacuum) pressure will be less than 150 mmHg due to the force of gravity.

Continuing the example above, if a first distance between the point of reference 112 and the elevation that is above the point of reference 112 is known and provided as an input to the ophthalmic surgical system 102, then the ophthalmic surgical system 102 is capable of adjusting the infusion pressure and the aspiration pressure to compensate for the first distance. For example, one or more processors of the ophthalmic surgical system 102 can execute instructions which cause an infusion subsystem to increase the infusion pressure and an aspiration subsystem to decrease the aspiration pressure such that the delivered infusion pressure will be 25 mmHg and the delivered aspiration pressure will be 150 mmHg at the elevation that is above the point of reference 112. Similarly, if a second distance between the point of reference 112 and the elevation that is below the point of reference 112 is known and provided as an input to the ophthalmic surgical system 102, then the ophthalmic surgical system 102 is capable of adjusting the infusion pressure and the aspiration pressure to compensate for the second distance. For example, the one or more processors of the ophthalmic surgical system 102 can execute instructions which cause the infusion subsystem to decrease the infusion pressure and the aspiration subsystem to increase the aspiration pressure such that the delivered infusion pressure will be 25 mmHg and the delivered aspiration pressure will be 150 mmHg at the elevation that is below the point of reference 112.

FIG. 1 further illustrates a patient 114 having a corresponding patient eye level (PEL) 116 which, in the example of FIG. 1, is below the point of reference 112. A vertical distance 120 between the point of reference 112 and the PEL 116 is shown in FIG. 1 relative to projections of the point of reference 112 and the PEL 116. Notably, the projections of the point of reference 112 and the PEL 116 are not available in a physical surgical environment but are included in FIG. 1 to illustrate the challenges of accurately measuring the vertical distance 120. In particular, the point of reference 112 depicted on the cassette 110 and the patient 114 do not share a vertical plane. As a result, in order to measure the vertical distance 120, a location of the PEL 116 may be estimated in a vertical plane that includes the point of reference 112 or a location of the point of reference 112 may be estimated in a vertical plane that includes the PEL 116. The vertical distance 120 may then be estimated/measured between the estimated location (which may be inaccurate) and the point of reference 112 or the PEL 116. However, even if the estimated location is accurate, the value estimated/measured for the vertical distance 120 will likely be inaccurate because of the difficulty in measuring between something which can be visually perceived (e.g., the point of reference 112 or the PEL 116) and something that cannot be visually perceived (e.g., the estimated location).

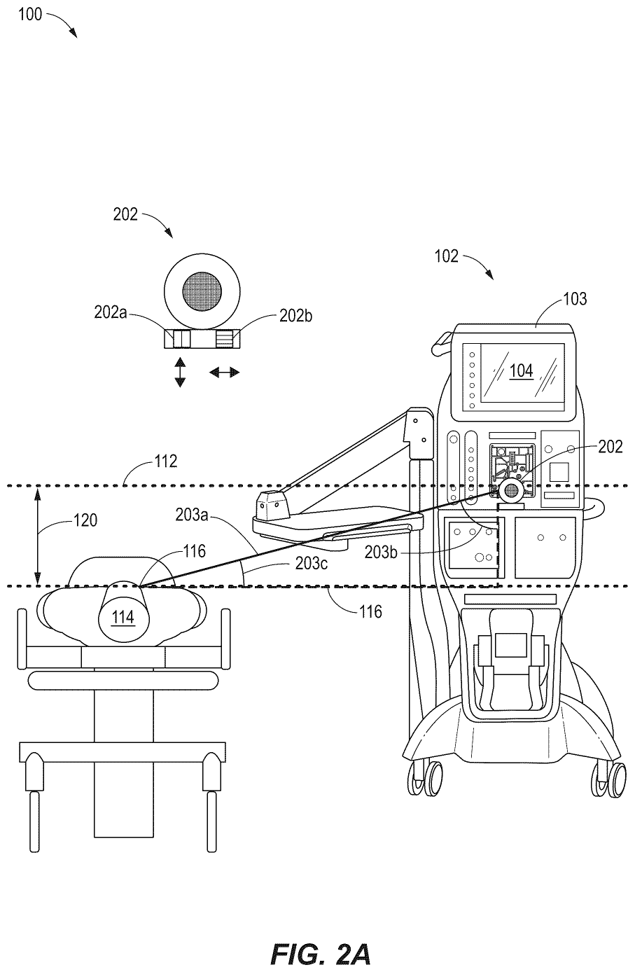

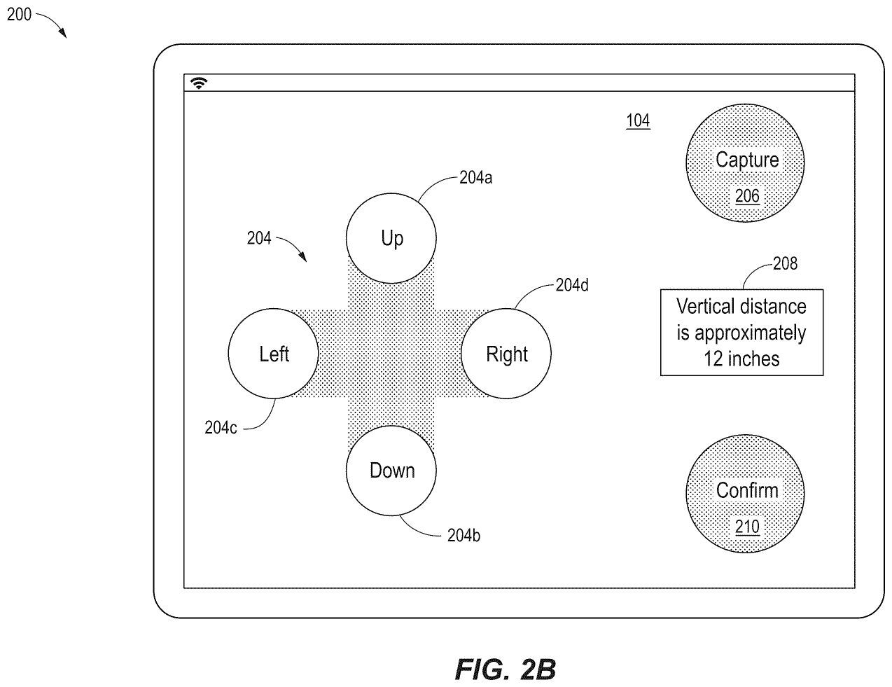

FIG. 2A illustrates a first example system for estimating a distance, according to embodiments described herein. In the embodiments of FIG. 2A, the console 103 includes a distance estimation device 202 configured for use in estimating the vertical distance 120 between the point of reference 112 and the PEL 116. In the illustrated example, the distance estimation device 202 is included in the console 103 adjacent to the cassette 110. For example, the distance estimation device 202 can be embedded in a portion of the ophthalmic surgical system 102. However, in some embodiments, the distance estimation device 202 is disposed on the tray 106 or included in a dedicated mechanical arm that is coupled to the console 103 such as the mechanical arm 108. In some embodiments, the distance estimation device 202 may be disposed on or adjacent to the patient 114.

In some embodiments, the distance estimation device 202 is configured to estimate the vertical distance 120 using light. For example, the distance estimation device 202 may include a laser level (or another laser device configured to propagate laser light) which estimates the vertical distance 120. In some embodiments, the distance estimation device 202 includes a laser and electromechanical components 202a, 202b. In some embodiments, the electromechanical component 202a includes a first actuator that is configured to actuate the laser of the laser level up or down. In some embodiments, the electromechanical component 202b includes a second actuator that is configured to actuate the laser of the laser level left or right.

In some embodiments, the distance estimation device 202 projects laser light (e.g., red laser light, green laser light, etc.) from the laser of the laser level to the PEL 116. In some embodiments, the electromechanical components 202a, 202b adjust an orientation of the distance estimation device 202 relative to the ophthalmic surgical system 102 to align the laser light with the PEL 116. When the laser light is aligned with the PEL 116, the laser level measures a distance 203a (d), an angle 203b (α), and/or an angle 203c (β). As shown, the distance 203a is representative of a hypotenuse of a right triangle, and the vertical distance 120 corresponds to an adjacent side relative to the angle 203b and an opposite side relative to the angle 203c. In some embodiments, the vertical distance 120 is computable as d*cos α and/or d*sin β.

After estimating the vertical distance 120, the distance estimation device 202 provides the vertical distance 120 as an input to the ophthalmic surgical system 102. In some embodiments, one or more processors of the ophthalmic surgical system 102 execute instructions which cause the infusion subsystem to adjust infusion pressure such that infusion pressure delivered to the PEL 116 matches an infusion pressure level specified by the user. In some embodiments, the instructions executed by the one more processors cause the aspiration subsystem to adjust aspiration pressure such that aspiration pressure delivered to the PEL 116 matches an aspiration pressure level specified by the user. Notably, the infusion pressure and the aspiration pressure are maintained after adjustment.

FIG. 2B illustrates a first example user interface 200 that may be used in conjunction with the distance estimation device 202 of FIG. 2A, according to embodiments described herein. In the illustrated example, the first example user interface 200 is displayed by the display device 104 to facilitate control of the laser level. As shown, the first example user interface 200 includes a user interface element 204 for adjusting an orientation of the laser level relative to the console 103. Adjusting the orientation of the laser level relative to the console 103 adjusts a direction of laser light emitting from the laser level relative to the PEL 116.

For example, a user interaction with user interface element 204a causes the electromechanical component 202a to actuate the orientation of the laser level up relative to the console 103 (and adjusts the direction of the laser light emitting from the laser level up relative to the PEL 116). A user interaction with user interface element 204b causes the electromechanical component 202a to actuate the orientation of the laser level down relative to the console 103 (and adjusts the direction of the laser light emitting from the laser level down relative to the PEL 116). A user interaction with user interface element 204c causes the electromechanical component 202b to actuate the orientation of the laser level towards the left relative to the console 103 (and adjusts the direction of the laser light emitting from the laser level towards the left relative to the PEL 116). A user interaction with user interface element 204d causes the electromechanical component 202b to actuate the orientation of the laser level towards the right relative to the console 103 (and adjusts the direction of the laser light emitting from the laser level towards the right relative to the PEL 116).

In some embodiments, when the laser light emitting from the laser level is aligned with the PEL 116 as illustrated in FIG. 2A, a user interaction with user interface element 206 causes the laser level to measure the distance 203a (d), the angle 203b (a) and/or the angle 203c (B). In some embodiments, the distance estimation device 202 and/or the one or more processors of the ophthalmic surgical system 102 compute the vertical distance 120 based on the distance 203a (d), the angle 203b (a) and/or the angle 203c (B). As shown, the one or more processors of the ophthalmic surgical system 102 execute instructions which cause the one or more processors to render an indication 208 for display by the display device 104. In the illustrated example, the indication 208 states “Vertical distance is approximately 12 inches.”

In some embodiments, the first example user interface 200 may include a user interface element 210 such that a user interaction with the user interface element 210 confirms that the vertical distance 120 is about 12 inches. In some embodiments, the user interaction with the user interface element 210 causes the one or more processors of the ophthalmic surgical system 102 to execute instructions which cause the infusion subsystem and the aspiration subsystem to adjust the infusion and aspiration pressure supplied to the PEL 116, respectively, to match infusion and aspiration pressure levels specified by the user of the ophthalmic surgical system 102. Since the PEL 116 is below the point of reference 112, the one or more processers can cause the infusion subsystem to decrease the infusion (irrigation) pressure by about 1.868 mmHg per inch of the 12 inches or by about 22.416 mmHg in total. Conversely, the one or more processors can cause the aspiration subsystem to increase the aspiration (vacuum) pressure by about 1.868 mmHg per inch of the 12 inches or by about 22.416 mmHg in total.

Note that although adjusting the orientation of the laser level relative to the console 103 is described with respect to the user interface element 204, it is to be appreciated that, in some embodiments, the orientation of the laser level relative to the console 103 is adjusted differently. In some embodiments, the orientation of the laser level relative to the console 103 may be adjusted by controlling the electromechanical components 202a, 202b using an electromechanical system (e.g., a joystick). In some embodiments, the orientation of the laser level relative to the console 103 can be adjusted manually (e.g., by the user of the user of the ophthalmic surgical system 102).

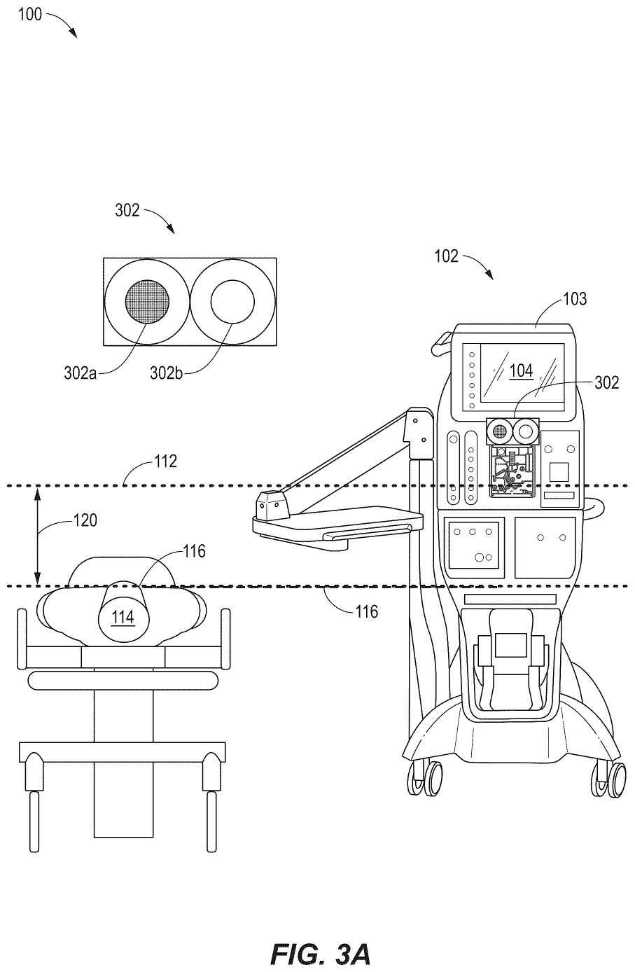

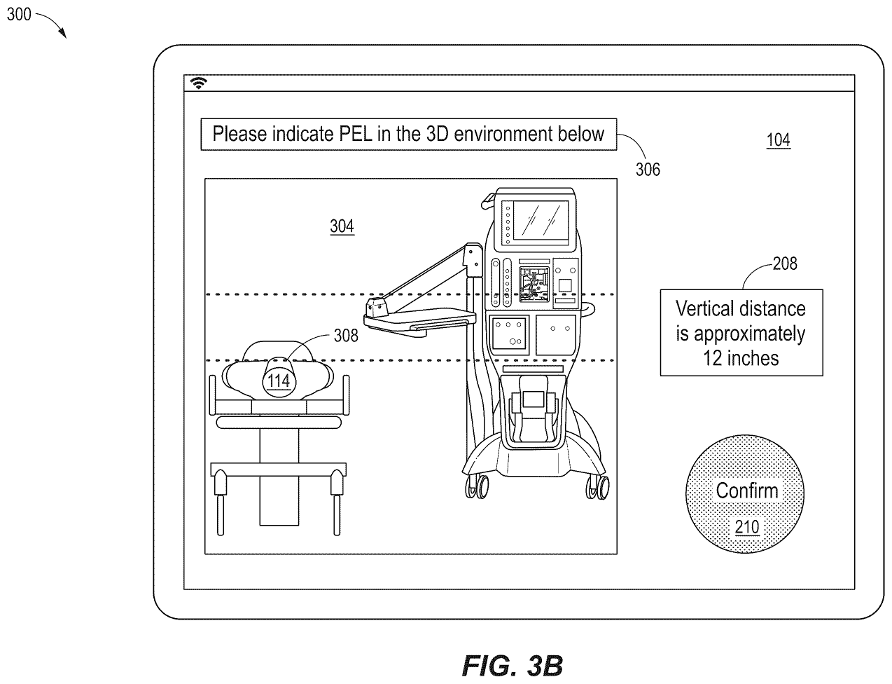

FIG. 3A illustrates a second example system for of estimating a distance, according to embodiments described herein. In the embodiments of FIG. 3A, the console 103 includes a distance estimation device 302 with a light detection and ranging (LIDAR) system which estimates the vertical distance 120 by three-dimensionally mapping the surgical environment 100. In the illustrated example, the distance estimation device 302 includes one light transmitter 302a and one light receiver 302b. Although, it is to be appreciated that the LIDAR system can include multiple light transmitters and multiple light receivers.

The LIDAR system maps the surgical environment 100 by transmitting light via the light transmitter 302a, detecting reflected light via the light receiver 302b, and measuring time between the transmission and the reflection of the light. In some embodiments, the distance estimation device 302 computes a three-dimensional map of the surgical environment 100 based on time between the transmission and the reflection of the light. After computing the three-dimensional map of the surgical environment 100, the distance estimation device 302 is capable of computing distances between any portions of the surgical environment 100 by computing distances between three-dimensional coordinates corresponding to the portions of the surgical environment 100.

In some embodiments, the distance estimation device 302 is not limited to estimating the vertical distance 120 using light. For example, the distance estimation device 302 includes a millimeter wavelength radar system which estimates the vertical distance 120 by three-dimensionally mapping the surgical environment 100. The millimeter wavelength radar system maps the surgical environment 100 by transmitting millimeter wavelength radar waves, detecting reflected millimeter wavelength radio waves, and measuring time between the transmission and the reflection of the millimeter wavelength radar waves.

FIG. 3B illustrates a second example user interface 300, according to embodiments described herein. As shown, the second example user interface 300 is displayed by the display device 104, which can facilitate distance estimation using the light detection and ranging (LIDAR) system and/or the millimeter wavelength radar system of FIG. 3A. In some embodiments, the second example user interface 300 includes a three-dimensional map 304 of the surgical environment 100 generated by the LIDAR system and/or the millimeter wavelength radar system.

An indication 306 is displayed by the display device 104 that states “Please indicate PEL in the 3D environment below.” In some embodiments, the user of the ophthalmic surgical system 102 interacts with the display device 104 relative to the three-dimensional map 304 to provide an indication 308 of a location of the PEL 116. In some embodiments, the user of the ophthalmic surgical system 102 may also indicate a location of the point of reference 112 or an elevation of the point of reference 112 can be predetermined by the one or more processors of the ophthalmic surgical system 102.

In some embodiments, the one or more processors of the ophthalmic surgical system 102 execute instructions which cause the one or more processors to project two-dimensional coordinates of the indication 308 into three-dimensional coordinates in the three-dimensional map (e.g., by ray casting). The instructions executed by the one or more processors cause the one or more processors to estimate the vertical distance 120 based on the three-dimensional coordinates projected according to the indication 308 and three-dimensional coordinates of the point of reference 112. In some embodiments, the one or more processors render the indication 208 for display by the display device 104 which states “Vertical distance is approximately 12 inches.” In some embodiments, the user of the ophthalmic surgical system 102 interacts with the user interface element 210 to confirm that the vertical distance 120 is about 12 inches. In various embodiments, the user's interaction with the user interface element 210 causes the one or more processors of the ophthalmic surgical system 102 to cause the infusion subsystem and the aspiration subsystem adjust the infusion and aspiration pressure supplied to the PEL 116, respectively, to match the infusion and aspiration pressure levels specified by the user of the ophthalmic surgical system 102.

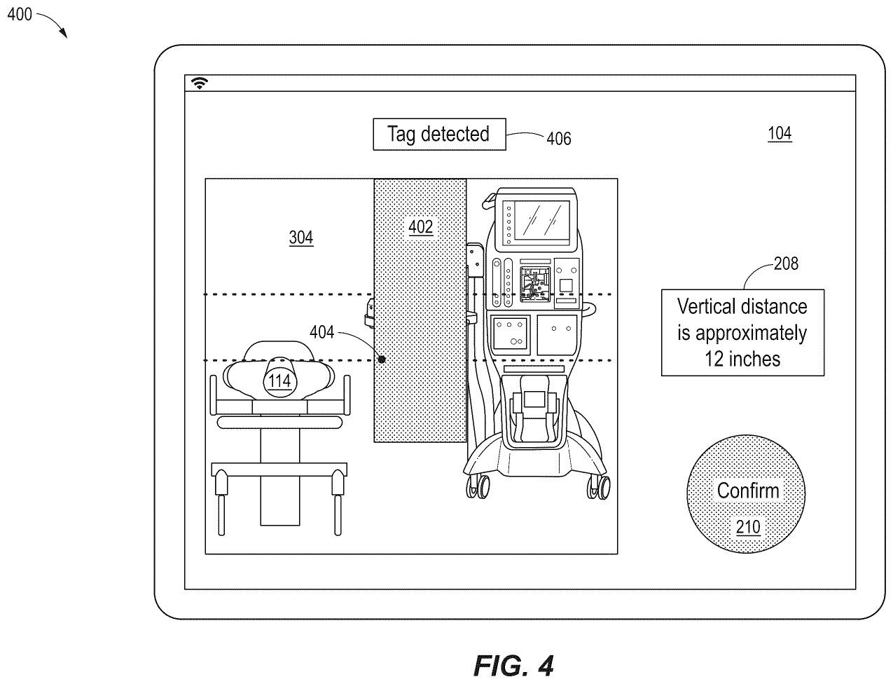

FIG. 4 illustrates a third example user interface 400, according to embodiments described herein. Similarly to the second example user interface 300, the third example user interface 400 facilitates distance estimation using the LIDAR system and/or the millimeter wavelength radar system described in relation to FIG. 3A. In the illustrated example, the third example user interface 400 includes the three-dimensional map 304 of the surgical environment 100 generated by the LIDAR system and/or the millimeter wavelength radar system. As shown in the three-dimensional map 304, the surgical environment 100 includes a drape 402 and a target such as a detectable tag 404 at the PEL 116. In some embodiments, the detectable tag 404 is a colored or marked sticker placed on the drape 402 at the PEL 116 before performing an ophthalmic surgical procedure and removed from the drape 402 after performing the ophthalmic surgical procedure. In some embodiments, the detectable tag 404 may include a barcode or a QR code. In some embodiments, the target includes a trocar cannula (e.g., inserted in an eye of the patient 114 in addition or alternative to the detectable tag 404). In some embodiments, the trocar cannula is detected to indicate the PEL 116.

In some embodiments, upon detecting the detectable tag 404, the one or more processors of the ophthalmic surgical system 102 execute instructions which cause the one or more processors to use a location of the detectable tag 404 and the location of the point of reference 112 to compute the vertical distance 120 and display an indication 406. As shown, the indication 406 states “Tag detected.” In some embodiments, the instructions executed by the one or more processors cause the one or more processors to render the indication 208 for display by the display device 104 which states “Vertical distance is approximately 12 inches.” In some embodiments, the user of the ophthalmic surgical system 102 interacts with the user interface element 210 to confirm that the vertical distance 120 is about 12 inches. In some embodiments, by interacting with the user interface element 210, the user causes the one or more processors of the ophthalmic surgical system 102 to cause the infusion subsystem and the aspiration subsystem to adjust the infusion and aspiration pressure supplied to the PEL 116, respectively, to match the infusion and aspiration pressure levels specified by the user of the ophthalmic surgical system 102.

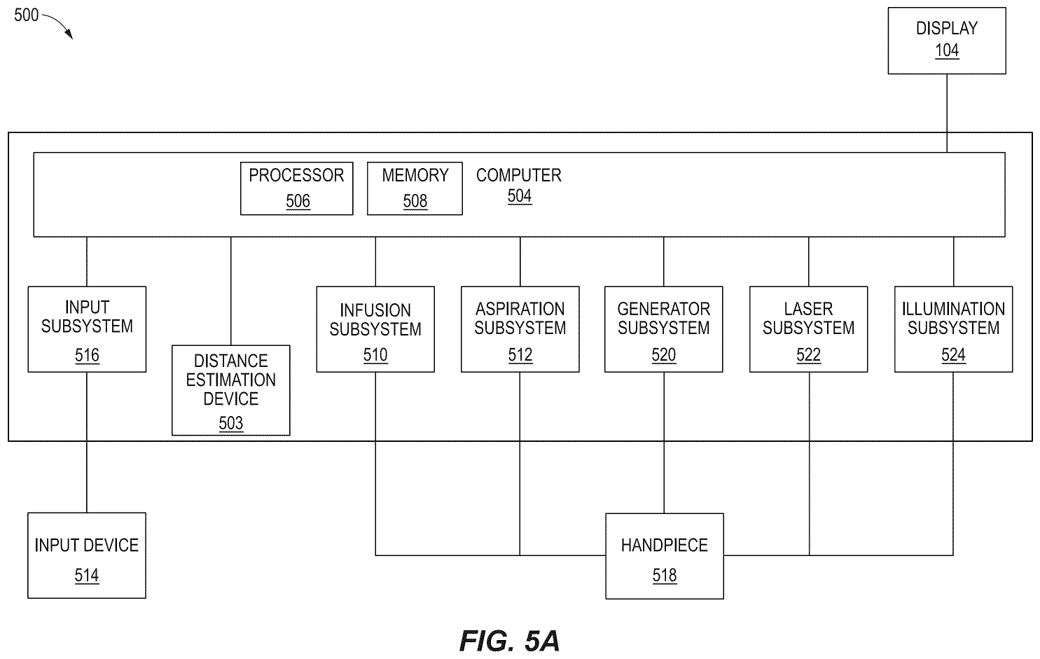

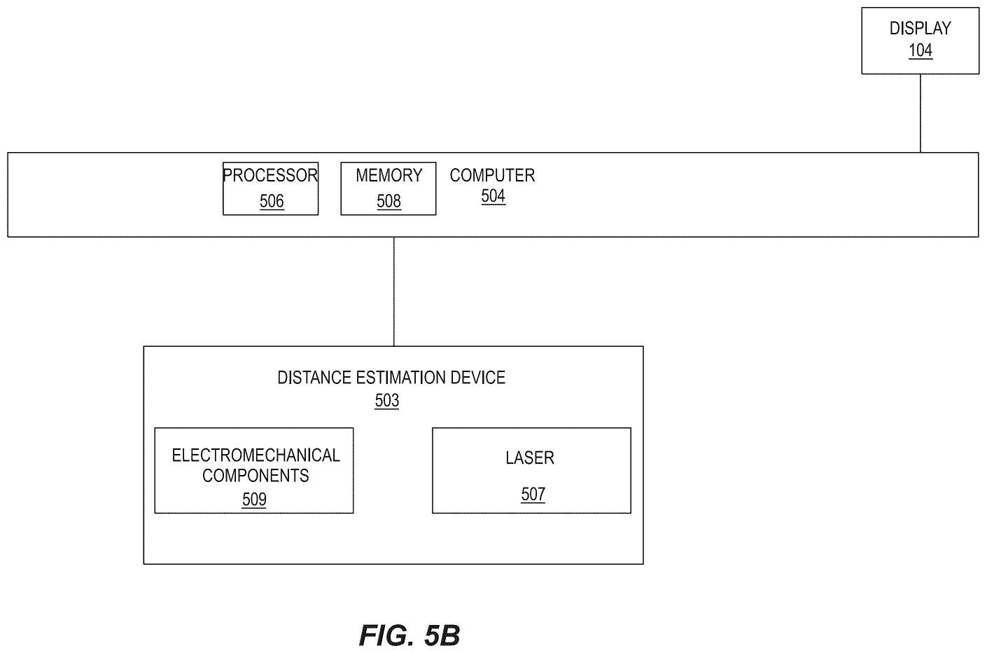

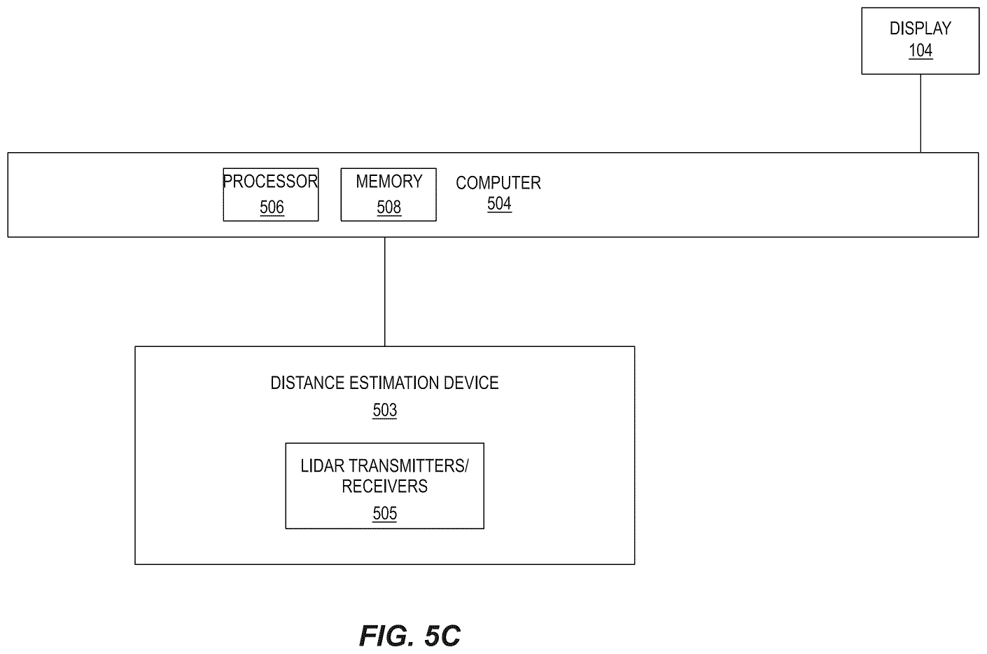

FIG. 5A illustrates an example of a surgical console 500, according to embodiments described herein. Surgical console 500 may be any of the surgical consoles illustrated and described in relation to FIGS. 2A and 3A. As shown, the surgical console 500 includes the display device 104 and a distance estimation device 503. The surgical console 500 is illustrated to include a computer 504 having a processor 506 and a memory 508. The processor 506 is representative of a variety of types of processors such as central processing units (CPUs), graphics processing units (GPUs), processors implemented using field-programmable gate arrays (FPGAs) (e.g., a soft processors), accelerators, etc. In some embodiments, the processor 506 includes multiple processors. The memory 508 includes volatile and/or non-volatile memory. In some embodiments, the memory 508 includes one or more computer readable media storing executable instructions.

In some embodiments, the distance estimation device 503 is communicatively coupled to the computer 504. The distance estimation device 503 communicates the vertical distance 120 to the computer 504, and the processor 506 executes instructions that cause the processor 506 to control an infusion subsystem 510 and an aspiration subsystem 512 based on the vertical distance 120. The infusion subsystem 510 controls the infusion pressure and the aspiration subsystem 512 controls the aspiration pressure. An input device 514 such as a footswitch is communicatively coupled to an input subsystem 516. A handpiece 518 is coupled to the infusion subsystem 510 and the aspiration subsystem 512 as well as a generator subsystem 520 (e.g., for RF diathermy), a laser subsystem 522 (e.g., for photocoagulation), and an illumination subsystem 524 (e.g., for intraocular illumination).

FIG. 5B illustrates an example schematic diagram of a distance estimation device 503 that includes electromechanical components 509 and a laser 507. In some embodiments, the distance estimation device 503 corresponds to the distance estimation device 202 of FIG. 2A. For example, the electromechanical components 509 include the electromechanical components 202a, 202b.

FIG. 5C illustrates an example schematic diagram of a distance estimation device 503 that includes LIDAR transmitters/receivers 505. In some embodiments, the distance estimation device 503 corresponds to the distance estimation device 302. In some embodiments, the LIDAR transmitters/receivers 505 include the light transmitter 302a and the light receiver 302b.

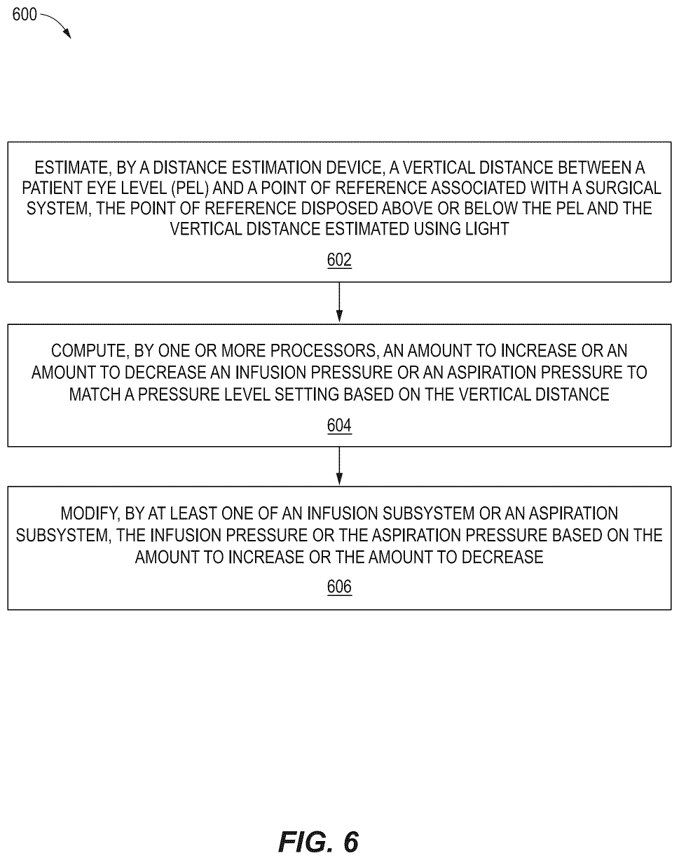

FIG. 6 illustrates an example method 600 for adjusting infusion pressure or aspiration pressure, according to embodiments described herein. At operation 602, a vertical distance between a patient eye level (PEL) and a point of reference associated with a surgical system is estimated by a distance estimation device using light, the point of reference disposed above or below the PEL. In some embodiments, the vertical distance 120 between the PEL 116 and the point of reference 112 on the ophthalmic surgical system 102 is estimated by the distance estimation device 202 using the laser level or by the distance estimation device 302 using the light detection and ranging (LIDAR) system.

At operation 604, an amount to increase or an amount to decrease an infusion pressure or an aspiration pressure to match a pressure level setting is computed by one or more processors of the surgical system based on the vertical distance. For example, the processor 506 computes the amount of infusion pressure for the infusion subsystem 510 to increase or decrease to match the infusion pressure level setting based on the vertical distance 120. Alternatively or additionally, the processor 506 computes the amount of aspiration pressure for the aspiration subsystem 512 to increase or decrease to match the aspiration pressure level setting based on the vertical distance 120.

At operation 606, the infusion pressure or the aspiration pressure is modified by the aspiration subsystem 512 based on the amount to increase or the amount to decrease. In some embodiments, the processor 506 executes instructions which cause the infusion subsystem 510 to modify the infusion pressure or the aspiration subsystem 512 to modify the aspiration pressure based on the amount to increase or the amount to decrease.

The disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

What is claimed is:

1. A surgical system comprising:
at least one of an infusion subsystem or an aspiration subsystem;
a distance estimation device configured to estimate a vertical distance between a point of reference associated with the surgical system and a patient eye level (PEL) using light, wherein:
the point of reference corresponds to a pressure level setting for infusion or aspiration, and
is above or below the PEL;
one or more processors configured to execute instructions that cause the one or more processors to:
receive the vertical distance; and
cause the at least one of the infusion subsystem or the aspiration subsystem to control an infusion pressure or an aspiration pressure based on the vertical distance to match the pressure level setting at the PEL.

2. The surgical system of claim 1, wherein:
the light includes laser light; and
the distance estimation device comprises a laser device configured to propagate the laser light.

3. The surgical system of claim 2, wherein the distance estimation device further comprises one or more electromechanical components to adjust an orientation of the distance estimation device.

4. The surgical system of claim 3, wherein the vertical distance is estimated based on a distance from the distance estimation device to the PEL and an angle of the laser light.

5. The surgical system of claim 1, wherein:
the vertical distance is estimated using light detection and ranging (LIDAR); and
the distance estimation device comprises a LIDAR system.

6. The surgical system of claim 5, wherein the vertical distance is estimated using a target.

7. The surgical system of claim 6, wherein the target includes at least one of a detectable tag or a trocar cannula.

8. The surgical system of claim 1, wherein the distance estimation device is embedded in a portion of the surgical system, disposed on a tray, or included in a mechanical arm.

9. A method comprising:
estimating, by a distance estimation device, a vertical distance between a patient eye level (PEL) and a point of reference associated with a surgical system, the point of reference disposed above or below the PEL and the vertical distance estimated using light;
computing, by one or more processors, an amount to increase or an amount to decrease an infusion pressure or an aspiration pressure to match a pressure level setting based on the vertical distance; and
modifying, by at least one of an infusion subsystem or an aspiration subsystem, the infusion pressure or the aspiration pressure based on the amount to increase or the amount to decrease.

10. The method of claim 9, further comprising maintaining, by at least one of an infusion subsystem or aspiration subsystem, the infusion pressure or the aspiration pressure after modifying the infusion pressure or the aspiration pressure.

11. The method of claim 9, wherein:
the light includes laser light; and
the distance estimation device comprises a laser device configured to propagate the laser light.

12. The method of claim 11, further comprising adjusting an orientation of the distance estimation device to estimate the vertical distance.

13. The method of claim 12, wherein the vertical distance is estimated based on a distance from the distance estimation device to the PEL and an angle of the light.

14. The method of claim 9, wherein:
the vertical distance is estimated using light detection and ranging (LIDAR); and
the distance estimation device comprises a LIDAR system.

15. The method of claim 14, wherein the vertical distance is estimated based on at least one of a detectable tag or a trocar cannula.
